SEQUENCE LISTING (9 sequences)

SEQ ID NO: 1 - DNA, 1020 bp, Helicobacter bizzozeronii
agtaaagagcaggttgaaaaggcttgaaaagggttgcgaaaagggagaaaatgcaaaaaaaagatatgcaaatgacaaagggagtgttgctttaaatcaaatatttacttttttgtgatgaaaatttctaggggataatgtgtaaaaatccttaactttggtgctttttattaaggagaatcagcatatggggtttatccagaacatgtttcgtcgtccactggttgtggtcttttttgtcttgttgagtgttgttggttgtgccaaaaagaatttcaatgaggctggtggcatgcattcttctatgggggatgcaaattttagtagtgctgtaagtggctcaacacaagattttatggtgaatgtgggagaccgtgttttctttagtcttaattcttcttctattgagcctgatgctgagcatgttttgaagcgtcaagccgagtggttgttgcgttatcctcattattttgttactgttgaagggcatgctgatgaccgtggaacacgcgaatataacttagcccttgggcagcgccgttctgttgctgttcgcgattatctcgtttctcttggggtttcttcacagcggatgagaacaatttcttatggaaaagaaagacctgttgctgtatgtgatgataattcatgttggaatcaaaatcggcgtgttgttttgacaatcaattccgtaaaggggggttaaatccataagcggtggattaaagccgtaggtggatggtggtcaaagaagtgaaatcaatggtctgttagagatcattaacaagtatttaggggagaaaagattttgcatgctgggcgcaaaaagagctggaaagttggttttttatatcaattatcattctggcatgcttgcgtttttacattaattatggtggtttgttttttggggcctgttcaaagtacacctcaaaatgctattcaattttctgatcacaatttaaattcatattatggtcacagtttgggcgcagggttgcccagagcaggatcacggga

SEQ ID NO: 2 - protein, 173 aa, Helicobacter bizzozeronii
  UNSURE (173): Xaa at position 173 is unknown or other
MGFIQNMFRRPLVVVFFVLLSVVGCAKKNFNEAGGMHSSMGDANFSSAVSGSTQDFMVNVGDRVFFSLNSSSIEPDAEHVLKRQAEWLLRYPHYFVTVEGHADDRGTREYNLALGQRRSVAVRDYLVSLGVSSQRMRTISYGKERPVAVCDDNSCWNQNRRVVLTINSVKGGX

SEQ ID NO: 3 - protein, 171 aa, Bartonella bacilliformis
MVWDFFANNAEYSYGNATPGSEQDFAITVGDRVFFSLDSSVIEADAQRILVRQAEWLILYPHHTIMIEGHADDRGTREYNLALGQRRAIAVRDYLVSLGVSPQRMKTMSYGKERPVAVCDDISCWNQNRRVVISITSGAVRKGFCQKSIFPIEKESFIAEKRAGRVNCLRA

SEQ ID NO: 4 - protein, 168 aa, Brucella abortus
MRRIQSIARSPIAIALFMSLAVAGCASKKNLPNNAGDLGLGAGAATPGSSQDFTVNVGDRIFFDLDSSLIRADAQQTLSKQAQWLQRYPQYSITIEGHADERGTREYNLALGQRRAAATRDFLASRGVPTNRMRTISYGNERPVAVCDADTCWSQNRRAVTVLNGAGR

SEQ ID NO: 5 - protein, 155 aa, Rickettsia prowazekii
MKTKITLAFLALFMLAGCNTTKKTTQMAGMVHQCEETSLIKDFEKHAGNAVWFAFDSSSLSPKAKEELERQACWLSKHPEVKVTVEGHCDERGTREYNLALGERRAAAAKQFLANKGIAHNRLNTISYGKDKPAMIGNTEEAFSYNRRAVTVLHK

SEQ ID NO: 6 - DNA, 22 bp, Artificial Sequence
  Description of Artificial Sequence  Primer
tatggggtttatccagaacatg

SEQ ID NO: 7 - DNA, 18 bp, Artificial Sequence
  Description of Artificial Sequence  Primer
gagctcacccccctttac

SEQ ID NO: 8 - DNA, 22 bp, Artificial Sequence
  Description of Artificial Sequence  Primer
ggatccatgtttcgtcgtccac

SEQ ID NO: 9 - DNA, 23 bp, Artificial Sequence
  Description of Artificial Sequence  Primer
gaattcttaacccccctttacgg

(19) United States
(12) Patent Application Publication
(10) Pub. No.: US 2005/0009037 A1
(43) Pub. Date: Jan. 13, 2005

(54) Helicobacter bizzozeronii outer membrane protein encoding gene and its use in diagnostic and treatment methods

(76) Inventors: Yung-Fu Chang, Ithaca, NY (US); Kenneth W. Simpson, Ithaca, NY (US); Jiaqian Zhu, Ithaca, NY (US)

Correspondence Address: Michael L. Goldman, Esq.;NIXON PEABODY LLP, Clinton Square, P.O. Box 31051, Rochester, NY, 14603, US

(21) Appl. No.: 10/639,387

(22) Filed: Aug. 12, 2003

Related U.S. Application Data
(60) Provisional application No. 60/404,340, filed on Aug. 16, 2002.

(51) Int. Cl. C12Q 1/68

(52) U.S. Cl. 435/6

(57) ABSTRACT

The present invention relates to an isolated nucleic acid molecule encoding an outer membrane protein of Helicobacter bizzozeronii. Also disclosed is the deduced protein or polypeptide sequence of the nucleic acid molecule of the present invention. Detection and treatment methods relating to H. bizzozeronii are also disclosed.

This application claims the benefit of U.S. Provisional Patent Application Ser. No. 60/404,340, filed Aug. 16, 2002.

### FIELD OF THE INVENTION

The present invention relates to an isolated nucleic acid molecule corresponding to an outer membrane protein gene of Helicobacter bizzozeronii, as well as to the use of the nucleic acid molecule and its corresponding protein or polypeptides in diagnostic and treatment methods.

### BACKGROUND OF THE INVENTION

Helicobacter bizzozeronii is a spiral, Gram-negative microaerophilic bacterium that was first cultured and characterized from gastric biopsies of canines (Hanninen et al., “Culture and Characteristics of Helicobacter bizzozeronii, a New Canine Gastric Helicobacter sp.,” Int. J. Syst. Bacteriol. 46:160-6 (1996)). H. bizzozeronii is 5 to 10 μm long and 0.3 μm wide with bipolar sheathed flagella and is indistinguishable morphologically from the Lockard type 3 bacterium and from Helicobacter heilmanii (Solnick et al., “Emergence of Diverse Helicobacter Species in the Pathogenesis of Gastric and Enterohepatic Diseases,” Clin. Microbiol. Rev. 14(1):59-97 (2001)). H. bizzozeronii is a slow growing organism whose colonies can be seen clearly on blood agar after about 8 to 10 days of incubation.

Although there are differences between H. bizzozeronii and other Helicobacter species, the phenotypic characteristics of the only human H. heilmanii isolate (R-53) to date are consistent with those of H. bizzozeronii, H. felis, and H. salomonis (Jalava et al., “A Cultured Strain of “Helicobacter heilmannii,” a Human Gastric Pathogen, Identified as H. bizzozeronii: Evidence for Zoonotic Potential of Helicobacter,” Emerg. Infect. Dis. 7:1036-1038 (2001)). H. bizzozeronii and other Helicobacter species are potential zoonotic agents (Jalava et al., “A Cultured Strain of “Helicobacter heilmannii,” a Human Gastric Pathogen, Identified as H. bizzozeronii: Evidence for Zoonotic Potential of Helicobacter,” Emerg. Infect. Dis. 7:1036-1038 (2001); Simpson et al., “The Relationship of Helicobacter spp. Infection to Gastric Disease in Dogs and Cats,” J. Vet. Intern. Med. 14:223-227 (2000); Simpson et al., “Helicobacter felis Infection in Dogs: Effect on Gastric Structure and Function,” Vet. Pathol. 36:237-248 (1999); Simpson et al., “Gastric Function in Dogs with Naturally Acquired Gastric Helicobacter spp. Infection,” J. Vet. Intern. Med. 13:507-515 (1999); Simpson et al., “Helicobacter felis Infection is Associated with Lymphoid Follicular Hyperplasia and Mild Gastritis but Normal Gastric Secretory Function in Cats,” Infect. Immun. 68:779-790 (2000); Simpson et al., “Helicobacter pylori Infection in the Cat: Evaluation of Gastric Colonization, Inflammation and Function,” Helicobacter 6:1-14 (2001); and Strauss-Ayali et al., “Gastric Helicobacter Infection in Dogs,” Vet. Clin. North. Am. Small. Anim. Pract. 29:397-414, vi (1999)). Furthermore, infection with H. heilmannii may be more frequently mucosa associated lymphoid tissue (MALT) lymphoma (Andersen et al., “Characterization of a Culturable “Gastrospirillum hominis” (Helicobacter heilmannii) Strain Isolated from Human Gastric Mucosa,” J. Clin. Microbiol. 37:1069-1076 (1999)).

Helicobacter species are known to play a role in gastric diseases such as ulcers and antral gastritis in both humans and animals. Due in part to the lack of definitive detection and diagnostic tools, H. bizzozeronii has not been studied as much as other Helicobacter species such as H. pylori, which is responsible for chronic active gastritis in people and animals (Solnick et al., “Emergence of Diverse Helicobacter Species in the Pathogenesis of Gastric and Enterohepatic Diseases,” Clin. Microbiol. Rev. 14(1):59-97 (2001)). Thus, knowledge of other Helicobacter species is important in understanding the role that H. bizzozeronii may play in various gastric conditions of canines and other animals, as well as in the development of diagnostic and treatment tools for the bacterium.

Surface-exposed proteins are constituents of the outer membrane in Gram-negative bacteria (Alm et al., “Comparative Genomics of Helicobacter pylori: Analysis of the Outer Membrane Protein Families,” Infect. Immun. 68:4155-4168 (2000)). The outer-membrane proteins are responsible for nutrient transportation and bacterial colonization (Alm et al., “Comparative Genomics of Helicobacter pylori: Analysis of the Outer Membrane Protein Families,” Infect. Immun. 68:4155-4168 (2000)), and may be important in other interactions with the environment and with the host. Also, outer-membrane proteins of pathogenic bacteria are potentially pivotal immunogens (Keenan et al., “Immune Response to an 18-Kilodalton Outer Membrane Antigen Identifies Lipoprotein 20 as a Helicobacter pylori Vaccine Candidate,” Infect. Immun. 68:3337-3343 (2000); Ge et al., “Characterization of Proteins in the Outer Membrane Preparation of a Murine Pathogen, Helicobacter bilis,” Infect. Immun. 69:3502-3506 (2001); Peck et al., “Characterization of Four Members of a Multigene Family Encoding Outer Membrane Proteins of Helicobacter pylori and Their Potential for Vaccination,” Microbes Infect. 3:171-179 (2001)).

Several families of outer-membrane protein (Omp) have been identified in H. pylori (Alm et al., “Comparative Genomics of Helicobacter pylori: Analysis of the Outer Membrane Protein Families,” Infect. Immun. 68:4155-4168 (2000)). One major family (Hop) contains thirty-two homologous members, some of which are functional as porins (Peck et al., “Characterization of Four Members of a Multigene Family Encoding Outer Membrane Proteins of Helicobacter pylori and Their Potential for Vaccination,” Microbes Infect. 3:171-179 (2001)). Some outer-membrane proteins not in paralogous families have been also identified. Among them are small-molecular-mass bacterial peptidoglycan-associated lipoproteins (PAL) such as Lpp20 and Omp22 (Kostrzynska et al., “Molecular Characterization of a Conserved 20-Kilodalton Membrane-Associated Lipoprotein Antigen of Helicobacter pylori,” J. Bacteriol. 176:5938-5948 (1994); Kim et al., “Cloning and Characterization of a 22 kDa Outer-Membrane Protein (Omp22) from Helicobacter pylori,” Mol. Cells 10:633-641 (2000)).

The present invention is directed to overcoming the deficiencies in the prior art.

### SUMMARY OF THE INVENTION

The present invention relates to an isolated nucleic acid molecule encoding an outer membrane protein of Helicobacter bizzozeronii.

The present invention also relates to isolated proteins or polypeptides encoded by the isolated nucleic acid molecule of the present invention. The isolated nucleic acid molecule can be inserted as heterologous DNA in an expression vector forming a recombinant DNA expression system for producing the proteins or polypeptides. Likewise, the heterologous DNA, usually inserted in an expression vector to form a recombinant DNA expression system, can be incorporated in a cell to achieve this objective.

The isolated protein or polypeptide of the present invention can be combined with a pharmaceutically-acceptable carrier to form a vaccine or used alone for administration to mammals, for preventing onset of disease resulting from infection by Helicobacter bizzozeronii. Alternatively, the protein or polypeptide of the present invention can be used to raise an antibody or a binding portion thereof. The antibody or binding portion thereof may be used alone or combined with a pharmaceutically-acceptable carrier to treat mammals already exposed to Helicobacter bizzozeronii to induce a passive immunity to prevent disease occurrence.

The protein or polypeptide of the present invention or the antibodies or binding portions thereof raised against them can also be utilized in a method for detection of Helicobacter bizzozeronii in a sample of tissue or body fluids. When the proteins or polypeptides are utilized, they are provided as an antigen. Any reaction with the antigen or the antibody is detected using an assay system which indicates the presence of Helicobacter bizzozeronii in the sample. Alternatively, Helicobacter bizzozeronii can be detected in such a sample by providing a nucleotide sequence of the isolated nucleic acid molecules of the present invention as a probe in a nucleic acid hybridization assay or a gene amplification detection procedure (e.g., using a polymerase chain reaction procedure). Any reaction with the probe is detected so that the presence of Helicobacter bizzozeronii in the sample is indicated.

Isolation of the nucleic acid molecules of the present invention constitutes a significant advance in the treatment and detection of such bacteria. It also provides the basis for a vaccine to prevent infection by Helicobacter bizzozeronii and a pharmaceutical agent for passive immunization for those mammals exposed to Helicobacter bizzozeronii. The proteins utilized in the vaccine, or used to produce the pharmaceutical agent, can be produced at high levels using recombinant DNA technology.

In diagnostic applications, the proteins or polypeptides of the present invention, as well as antibodies and binding portions thereof against them, permit rapid determination of whether a particular individual mammal is infected with Helicobacter bizzozeronii. Moreover, such detection can be carried out without requiring an examination of the individual mammal being tested for an antibody response.

### BRIEF DESCRIPTION OF THE DRAWINGS

FIG. 1 shows the nucleotide sequence (SEQ ID NO:1) of the outer-membrane-protein gene and the predicted amino acid sequence (SEQ ID NO:2) of the corresponding protein. Promoter-like regions proximal to lip18 are indicated by bold and italics. Potential ribosome-binding sequences preceding lip18 are underlined, and the transcription start site of lip18 is bolded. A potential transcription terminator is indicated by ----> <--------.

FIG. 2 shows the alignment of the predicted amino acid sequences of Lip 18. The amino acid sequences were predicted from the known nucleotide sequences of outer-membrane proteins (H. bizzozeronii, H. bizzo; Brucella abortus, B. abortus (Genebank number, AAA59360), Bartonella bacilliformis, B. bacill, Genbank number, AAA78948), Agrobacterium tumefaciens, Agrobact, Genbank number, AAL44523, H. pylori, H. pylori, Genbank number, NP223771 and U75869). The predicted amino acid sequences of the Lip18 proteins of FIG. 2 are identified as follows (organism source is indicated in parentheses): “H. biz omp” (H. bizzozeronii) (SEQ ID NO:2); “B. bacil” (Bartonella bacilliformis) (SEQ ID NO:3); “B. abortus” (Brucella abortus) (SEQ ID NO:4); and “R. prowazek.” (Rickettsia prowazekii) (SEQ ID NO:5).

FIGS. 3A-3C show an SDS-PAGE (12% polyacrylamide) and Western-blot analyses of whole-cell extracts of H. bizzozeronii, and E. coli cells BL21 (DE3)LysS harboring the recombinant plasmids using rabbit antiserum raised against H. bizzozeronii Lip18. Each lane was loaded with about 20 μg of sample. The sample was prepared from the pellet of 1 mL of culture that was induced with 1 mM IPTG at OD₆₀₀=0.5 and incubated until OD₆₀₀=2.5. The pellet was suspended in 200 μL sample buffer, boiled for 5 min. and centrifuged for 2 min before loading. FIG. 3A: (1) E. coli with vector pET22b(+), (2) E. coli with pETLip18, Histidine-tagged Lip18 fusion protein. FIG. 3B: (1). E. coli with vector pGEGX4T-2, (2) E. coli with pGSTLip18, GST-tagged fusion protein. FIG. 3C: Western blot: (1). Vector pET22b(+), (2). Wild-type H. bizzozeronii. (3). Histidine-tagged Omp fusion protein. Molecular mass markers (in kilodaltons) are indicated (left). Arrows indicate the Lip18 fusion protein.

FIG. 4 shows the outer-membrane profiles of H. bizzozeronii on an SDS-12% polyacrylamide gel. 1: outer-membrane proteins, 2: inner-membrane proteins, 3: whole-cell lysates. Each lane was loaded with approximate 20 μg of protein.

FIG. 5 shows the Western-blot analysis of H. bizzozeronii Lip18. 1: outer-membrane-protein fraction, 2: inner-membrane-protein fraction, 3: whole-cell lysates. Each lane was loaded with approximate 20 μg of protein.

FIG. 6 shows the Western-blot analysis of H. bizzozeronii Ompl 8 against sera from dogs infected with Helicobacter like strains. 1, 2 and 3: sera from uninfected and SPF dogs; 4, 5, 6, 7, 8 and 9: sera from Helicobacter-like infected dogs.

FIG. 7 shows the Western blot analysis of H. bizzozeronii Lip18 against sera from cats infected with Helicobacter-like strains. 1: sera from uninfected cat; 2, 3, 4, 5, 6: sera from Helicobacter-like infected cats.

### DETAILED DESCRIPTION OF THE INVENTION

The present invention relates to the isolation and identification of a nucleic acid molecule that encodes an outer membrane protein from Helicobacter bizzozeronii. In one embodiment, the nucleic acid molecule of the present invention is a gene of H. bizzozeronii that is a homolog of the peptidoglycan-associated lipoproteins (PALs) of Helicobacter pylori. One form of the nucleic acid molecule of the present invention is a gene encoding the Lip18 outer membrane protein of H. bizzozeronii, which gene has a nucleotide sequence corresponding to SEQ ID NO: 1 as follows:

| | |
|---|---|
| agtaaagagc aggttgaaaa ggcttgaaaa gggttgcgaa aagggagaaa atgcaaaaaa | 60 |
| aagatatgca aatgacaaag ggagtgttgc tttaaatcaa atatttactt ttttgtgatg | 120 |
| aaaatttcta ggggataatg tgtaaaaatc cttaactttg gtgcttttta ttaaggagaa | 180 |
| tcagcatatg gggtttatcc agaacatgtt tcgtcgtcca ctggttgtgg tcttttttgt | 240 |
| cttgttgagt gttgttggtt gtgccaaaaa gaatttcaat gaggctggtg gcatgcattc | 300 |
| ttctatgggg gatgcaaatt ttagtagtgc tgtaagtggc tcaacacaag attttatggt | 360 |
| gaatgtggga gaccgtgttt tctttagtct taattcttct tctattgagc ctgatgctga | 420 |
| gcatgttttg aagcgtcaag ccgagtggtt gttgcgttat cctcattatt ttgttactgt | 480 |
| tgaagggcat gctgatgacc gtggaacacg cgaatataac ttagcccttg ggcagcgccg | 540 |
| ttctgttgct gttcgcgatt atctcgtttc tcttggggtt tcttcacagc ggatgagaac | 600 |
| aatttcttat ggaaaagaaa gacctgttgc tgtatgtgat gataattcat gttggaatca | 660 |
| aaatcggcgt gttgttttga caatcaattc cgtaaagggg ggttaaatcc ataagcggtg | 720 |
| gattaaagcc gtaggtggat ggtggtcaaa gaagtgaaat caatggtctg ttagagatca | 780 |
| ttaacaagta tttaggggag aaaagatttt gcatgctggg cgcaaaaaga gctggaaagt | 840 |
| tggtttttta tatcaattat cattctggca tgcttgcgtt tttacattaa ttatggtggt | 900 |
| ttgttttttg gggcctgttc aaagtacacc tcaaaatgct attcaatttt ctgatcacaa | 960 |
| tttaaattca tattatggtc acagtttggg cgcagggttg cccagagcag gatcacggga | 1020 |

The present invention also relates to a protein or polypeptide encoded by the nucleic acid molecule of the present invention. In one embodiment, the protein or polypeptide is Lip18, which is encoded by nucleotide bases 188 through 704 of the nucleotide sequence corresponding to SEQ ID NO: 1, where the encoded protein or polypeptide has an amino acid sequence corresponding to SEQ ID NO: 2 as follows:

```
Met Gly Phe Ile Gln Asn Met Phe Arg Arg Pro Leu Val Val Val Phe
    1 5 10 15
Phe Val Leu Leu Ser Val Val Gly Cys Ala Lys Lys Asn Phe Asn Glu
                          20 25 30
Ala Gly Gly Met His Ser Ser Met Gly Asp Ala Asn Phe Ser Ser Ala
                  35 40 45
Val Ser Gly Ser Thr Gln Asp Phe Met Val Asn Val Gly Asp Arg Val
          50 55 60
Phe Phe Ser Leu Asn Ser Ser Ser Ile Glu Pro Asp Ala Glu His Val
  65 70 75 80
Leu Lys Arg Gln Ala Glu Trp Leu Leu Arg Tyr Pro His Tyr Phe Val
                                  85 90 95
Thr Val Glu Gly His Ala Asp Asp Arg Gly Thr Arg Glu Tyr Asn Leu
                        100 105 110
Ala Leu Gly Gln Arg Arg Ser Val Ala Val Arg Asp Tyr Leu Val Ser
                115 120 125
Leu Gly Val Ser Ser Gln Arg Met Arg Thr Ile Ser Tyr Gly Lys Glu
        130 135 140
Arg Pro Val Ala Val Cys Asp Asp Asn Ser Cys Trp Asn Gln Asn Arg
145 150 155 160
Arg Val Val Leu Thr Ile Asn Ser Val Lys Gly Gly Xaa
                                  165 170
```

This protein or polypeptide has an estimated molecular weight of approximately 18.0 kilodaltons, based on the deduced amino acid sequence.

Also suitable as an isolated nucleic acid molecule according to the present invention is an isolated nucleic acid molecule including at least 20 contiguous nucleic acid residues that hybridize to a nucleic acid having a nucleotide sequence of SEQ ID NO: 1, or the complement of SEQ ID NO: 1 under stringent conditions. Homologous nucleotide sequences can be detected by selectively hybridizing to each other. The term “selectively hybridizing” is used herein to mean hybridization of DNA or RNA probes from one sequence to the “homologous” sequence under stringent conditions which are characterized by a hybridization buffer comprising 2×SSC, 0.1% SDS at 56° C. (Ausubel et al., eds., Current Protocols in Molecular Biology, Vol. 1, New York: Greene Publishing Associates, Inc. and John Wiley & Sons, Inc., p. 2.10.3 (1989), which is hereby incorporated by reference in its entirety). Another example of suitable stringency conditions is when hybridization is carried out at 65° C. for 20 hours in a medium containing 1 M NaCl, 50 mM Tris-HCl, pH 7.4, 10 mM EDTA, 0.1% sodium dodecyl sulfate, 0.2% ficoll, 0.2% polyvinylpyrrolidone, 0.2% bovine serum albumin, 50 μg/ml E. coli DNA. In one embodiment, the present invention is directed to isolated nucleic acid molecules having nucleotide sequences containing at least 20 contiguous nucleic acid residues that hybridize to the nucleic acid molecules of the present invention, including, SEQ ID NO: 1 under stringent conditions including 50 percent formamide at 42° C.

Fragments of the above proteins or polypeptides are encompassed by the present invention.

The proteins or polypeptides of the present invention are preferably produced in purified form by conventional techniques. To isolate the proteins or polypeptides, a protocol involving a host cell such as Escherchia coli may be used, in which protocol the E. coli host cell carrying a recombinant plasmid is propagated, homogenized, and the homogenate is centrifuged to remove bacterial debris. The supernatant is then subjected to sequential ammonium sulfate precipitation. The fraction containing the proteins or polypeptides of the present invention are subjected to gel filtration in an appropriately sized dextran or polyacrylamide column to separate the proteins or polypeptides. If necessary, the protein fraction may be further purified by high performance liquid chromatography (“HPLC”).

Fragments of the proteins or polypeptides of the present invention can be produced by digestion of a full-length elicitor protein with proteolytic enzymes like chymotrypsin or Staphylococcus proteinase A, or trypsin. Different proteolytic enzymes are likely to cleave the proteins or polypeptides of the present invention at different sites based on the amino acid sequence of the proteins or polypeptides. Some of the fragments that result from proteolysis may be active elicitors of resistance.

In another approach, based on knowledge of the primary structure of the protein or polypeptide, fragments of the genes encoding the proteins or polypeptides of the present invention may be synthesized by using the polymerase chain reaction (“PCR”) technique together with specific sets of primers chosen to represent particular portions of the protein or polypeptide of interest. These then would be cloned into an appropriate vector for expression of a truncated peptide or protein.

Chemical synthesis can also be used to make suitable fragments. Such a synthesis is carried out using known amino acid sequences for the protein or polypeptide being produced. Alternatively, subjecting a full length protein or polypeptide of the present invention to high temperatures and pressures will produce fragments. These fragments can then be separated by conventional procedures (e.g., chromatography, SDS-PAGE).

Variants may also (or alternatively) be made, for example, by the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

The protein or polypeptide of the present invention is preferably produced in purified form (preferably at least about 80%, more preferably 90%, pure) by conventional techniques. Typically, the protein or polypeptide of the present invention is secreted into the growth medium of Helicobacter cells or host cells which express a functional type III secretion system capable of secreting the protein or polypeptide of the present invention. Alternatively, the protein or polypeptide of the present invention is produced but not secreted into growth medium of recombinant host cells (e.g., Escherichia coli). In such cases, to isolate the protein, the host cell (e.g., E. coli) carrying a recombinant plasmid is propagated, lysed by sonication, heat, differential pressure, or chemical treatment, and the homogenate is centrifuged to remove bacterial debris. The supernatant is then subjected to sequential ammonium sulfate precipitation. The fraction containing the polypeptide or protein of the present invention is subjected to gel filtration in an appropriately sized dextran or polyacrylamide column to separate the proteins. If necessary, the protein fraction may be further purified by HPLC.

The DNA molecule encoding the proteins or polypeptides of the present invention can be incorporated in cells using conventional recombinant DNA technology. Generally, this involves inserting the DNA molecule into an expression system to which the DNA molecule is heterologous (i.e., not normally present). The heterologous DNA molecule is inserted into the expression system or vector in sense orientation and correct reading frame. The vector contains the necessary elements for the transcription and translation of the inserted protein-coding sequences. Thus, the present invention also relates to a DNA construct containing the nucleic acid of the present invention, which is operably linked to both a 5′ promoter and a 3′ regulatory region (i.e., transcription terminator) capable of affording transcription and expression of the encoded proteins or polypeptides of the present invention in host cells or host organisms.

The present invention also relates to an expression vector containing a DNA molecule encoding the proteins or polypeptides of the present invention. The nucleic acid molecules of the present invention may be inserted into any of the many available expression vectors using reagents that are well known in the art. In preparing a DNA vector for expression, the various DNA sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. The selection of a vector will depend on the preferred transformation technique and target host for transformation.

Suitable vectors for practicing the present invention include, but are not limited to, the following viral vectors such as lambda vector system gt11, gtWES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993)), pQE, pIH821, pGEX, pET series (Studier et al, “Use of T7 RNA Polymerase to Direct Expression of Cloned Genes,” Methods in Enzymology. 185:60-89 (1990) which is hereby incorporated by reference in its entirety), and any derivatives thereof. Any appropriate vectors now known or later described for genetic transformation are suitable for use with the present invention. Recombinant molecules can be introduced into cells via transformation, particularly transduction, conjugation, mobilization, or electroporation. The DNA sequences are cloned into the vector using standard cloning procedures in the art, as described by Maniatis et al., Molecular Cloning: A Laboratory Manual, Cold Springs Harbor, N.Y.: Cold Springs Laboratory, (1982), which is hereby incorporated by reference in its entirety.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is generally desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promotor, lac promotor, trp promotor, recA promotor, ribosomal RNA promotor, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promotor or other E. coli promoters produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

In one aspect of the present invention, the nucleic acid molecule of the present invention is incorporated into an appropriate vector in the sense direction, such that the open reading frame is properly oriented for the expression of the encoded protein under control of a promoter of choice. This involves the inclusion of the appropriate regulatory elements into the DNA-vector construct. These include non-translated regions of the vector, useful promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

A constitutive promoter is a promoter that directs expression of a gene throughout the development and life of an organism.

An inducible promoter is a promoter that is capable of directly or indirectly activating transcription of one or more DNA sequences or genes in response to an inducer. In the absence of an inducer, the DNA sequences or genes will not be transcribed.

The DNA construct of the present invention also includes an operable 3′ regulatory region, selected from among those which are capable of providing correct transcription termination and polyadenylation of mRNA for expression in the host cell of choice, operably linked to a DNA molecule which encodes for a protein of choice.

The vector of choice, promoter, and an appropriate 3′ regulatory region can be ligated together to produce the DNA construct of the present invention using well known molecular cloning techniques as described in Sambrook et al., Molecular Cloning: A Laboratory Manual, Second Edition, Cold Spring Harbor Press, NY (1989), and Ausubel, F. M. et al. Current Protocols in Molecular Biology, New York, N.Y.: John Wiley & Sons, (1989), which are hereby incorporated by reference in their entirety.

Once the DNA construct of the present invention has been prepared, it is ready to be incorporated into a host cell. Accordingly, another aspect of the present invention relates to a method of making a recombinant cell. Basically, this method is carried out by transforming a host cell with a DNA construct of the present invention under conditions effective to yield transcription of the DNA molecule in the host cell. Recombinant molecules can be introduced into cells via transformation, particularly transduction, conjugation, mobilization, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable host cells include, but are not limited to, bacteria, virus, yeast, mammalian cells, insect, plant, and the like.

Generally, the mammalian immune system responds to infection by pathogenic bacteria by producing antibodies that bind to specific proteins or carbohydrates on the bacterial surface. The antibodies stimulate binding to macrophages which have receptors that bind to the F<c> region of the antibodies. Other serum proteins, called complement, coat the foreign particle and stimulate their ingestion by binding to specific surface receptors on the macrophage. Once the particle is bound to the surface of the macrophage, the sequential process of ingestion begins by continual apposition of a segment of the plasma membrane to the particle surface. Surface receptors on the membranes then interact with ligands distributed uniformity over the particle surface to link the surfaces together. The macrophage enveloping the particle is then delivered to lysosomes where the particle is ingested.

In view of the present invention's determination of a nucleotide sequence conferring on Helicobacter bizzozeronii an ability to produce an outer membrane protein, a wide array of therapeutic and/or prophylatic agents and diagnostic procedures for, respectively, treating and detecting Helicobacter bizzozeronii can be developed.

For example, an effective amount of the proteins or polypeptides of the present invention can be administered alone or in combination with a pharmaceutically-acceptable carrier to mammals such as canines and felines, as a vaccine, for preventing onset of disease resulting from infection by Helicobacter bizzozeronii. Alternatively, it is possible to administer to individuals exposed to Helicobacter bizzozeronii an effective amount of an antibody or binding portion thereof against these proteins or polypeptides as a passive immunization. Such antibodies or binding portions thereof are administered alone or in combination with a pharmaceutically-acceptable carrier to effect short term treatment of individuals who may have been recently exposed to Helicobacter bizzozeronii.

Antibodies suitable for use in inducing passive immunity can be monoclonal or polyclonal.

Monoclonal antibody production may be effected by techniques which are well-known in the art. Basically, the process involves first obtaining immune cells (lymphocytes) from the spleen of a mammal (e.g., mouse) which has been previously immunized with the antigen of interest (i.e., the protein or peptide of the present invention) either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference in its entirety.

Mammalian lymphocytes are immunized by in vivo immunization of the animal (e.g., a mouse) with one of the proteins or polypeptides of the present invention. Such immunizations are repeated as necessary at intervals of up to several weeks to obtain a sufficient titer of antibodies. The virus is carried in appropriate solutions or adjuvants. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (PEG) or other fusing agents (See Milstein and Kohler, Eur. J. Immunol. 6:511 (1976), which is hereby incorporated by reference in its entirety). This immortal cell line, which is preferably murine, but may also be derived from cells of other mammalian species, including but not limited to rats and humans, is selected to be deficient in enzymes necessary for the utilization of certain nutrients, to be capable of rapid growth and to have good fusion capability. Many such cell lines are known to those skilled in the art, and others are regularly described.

Procedures for raising polyclonal antibodies are also well known. Typically, such antibodies can be raised by administering one of the proteins or polypeptides of the present invention subcutaneously to New Zealand white rabbits which have first been bled to obtain pre-immune serum. The antigens can be injected at a total volume of 100 [mu] 1 per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbitol 150 mg/Kg IV. This and other procedures for raising polyclonal antibodies are disclosed in E. Harlow, et. al., editors, Antibodies: A Laboratory Manual (1988), which is hereby incorporated by reference in its entirety.

In addition to utilizing whole antibodies, the processes of the present invention encompass use of binding portions of such antibodies. Such antibody fragments can be made by conventional procedures, such as proteolytic fragmentation procedures, as described in J. Goding, Monoclonal Antibodies: Principles and Practice, pp. 98-118 (N.Y. Academic press 1983), which is hereby incorporated by reference in its entirety.

The vaccines and passive immunization agents of this invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The solid unit dosage forms can be of the conventional type. The solid form can be a capsule, such as an ordinary gelatin type containing the proteins or peptides of the present invention or the antibodies or binding portions thereof of the present invention and a carrier, for example, lubricants and inert fillers such as, lactose, sucrose, or cornstarch. In another embodiment, these compounds are tableted with conventional tablet bases such as lactose, sucrose, or corn starch in combination with binders like acacia, corn starch, or gelatin, disintegrating agents such as, corn starch, potato starch, or alginic acid, and a lubricant like stearic acid or magnesium stearate.

The isolated nucleic acid molecules, proteins, or polypeptides of the present invention or the antibodies or binding portions raised against the proteins or polynucleotides of this invention may also be administered in injectable dosages by solution or suspension of these materials in a physiologically acceptable diluent with a pharmaceutical carrier. Such carriers include sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions.

The present invention also relates to a method of vaccinating mammals against onset of disease caused by infection of Helicobacter bizzozeronii. This method involves administering to mammals an effective amount of at least one of the isolated nucleic acid molecules of the present invention. Suitable techniques for such gene therapy techniques are well known and are described in U.S. Pat. Nos. 5,328,470 and 6,339,068, the entire disclosures of which are hereby incorporated by reference.

For use as aerosols, the proteins or polypeptides of the present invention or the antibodies or binding portions thereof of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In yet another aspect of the present invention, the proteins or polypeptides of the present invention can be used as antigens in diagnostic assays for the detection of Helicobacter bizzozeronii in body fluids. Alternatively, the detection of that bacterium can be achieved with a diagnostic assay employing antibodies or binding portions thereof raised by such antigens. Such techniques permit detection of Helicobacter bizzozeronii in a sample of the following tissue or body fluids: blood, spinal fluid, sputum, pleural fluids, urine, bronchial alveolor lavage, lymph nodes, bone marrow, or other biopsied materials.

In one embodiment, the assay system has a sandwich or competitive format. Examples of suitable assays include an enzyme-linked immunosorbent assay, a radioimmunoassay, a gel diffusion precipitan reaction assay, an immunodiffusion assay, an agglutination assay, a fluorescent immunoassay, a protein A immunoassay, or an immunoelectrophoresis assay.

In an alternative diagnostic embodiment of the present invention, the nucleotide sequences of the isolated DNA molecules of the present invention may be used as a probe in nucleic acid hybridization assays for the detection of Helicobacter bizzozeronii in various body fluids. The nucleotide sequences of the present invention may be used in any nucleic acid hybridization assay system known in the art, including, but not limited to, Southern blots (Southern, J. Mol. Biol. 98: 503-517 (1975) (which discloses hybridization in 2×SSC (i.e., 0.15M NaCl, 0.015 sodium citrate), 40% formamide at 40 degrees Celsius; Northern blots (Thomas et al., Proc. Nat'l Acad. Sci. USA 77:5201-05 (1980)); Colony blots (Grunstein et al. Proc. Nat'l Acad. Sci. USA 72:3961-65 (1975), which are hereby incorporated by reference in their entirety). Alternatively, the isolated DNA molecules of the present invention can be used in a gene amplification detection procedure (e.g., a polymerase chain reaction). See H. A. Erlich et. al., “Recent Advances in the Polymerase Chain Reaction,” Science 252:1643-51 (1991), which is hereby incorporated by reference in its entirety.

### EXAMPLES

The Examples set forth below are for illustrative purposes only and are not intended to limit, in any way, the scope of the present invention.

### Example 1

### Bacterial and Culture Conditions

A Helicobacter bizzozeronii strain, obtained from American Type Cell Culture (ATCC700030), was cultured on 5% sheep-blood agar plates in a microaerobic gas-generating system (Mitsubishi Gas Chemical Company, Inc. Japan) for 8 to 10 days. E. coli XL1-blue MRF′ XL1, SOLR, Topo10 and BL21 (DE3) plysS (Stratagene, Calif.) were grown in Luria broth (LB) and on LB agar with appropriate antibiotics (ampicillin 50 μg/mL, kanamycin 50 μg/mL, and chloramphenicol 34 μg/mL).

### Example 2

### Phage Library Screening for omp

The bacteriophage library of H. bizzozeronii (Zhu et al., “Cloning and Characterization of a Helicobacter bizzozeronii Urease Gene Cluster,” DNA Seq. (Submitted 2002), which is hereby incorporated by reference in its entirety) was immuno-screened using sera from a cat naturally infected with Helicobacter spp. Plaques giving positive signals were picked, re-screened and amplified on E. coli SOLR. Phages isolated from the positive plaques were excised as Bluescript plasmid according to the manufacturer's directions (Stratagene, Calif.).

### Example 3

### DNA Manipulation

To subclone the outer membrane protein (“omp”) gene, a primer pair was designed: sense: 5′-TATGGGGTTTATCCAGAACATG (SEQ ID NO:6), and anti-sense: GAGCTCACCCCCCTTTAC (SEQ ID NO:7). Polymerase chain reaction was carried out in a Perkin Elmer Gene Amp PCR system 9600 Thermal Cycler. The final volume for the PCR reaction was 50 μL. PWO polymerase (Roche diagnostics GmbH, Germany) was used. The amplified DNA fragment of 500 bp was ligated into a pCR-Blunt vector (Invitrogen, Carlsbad, Calif.) and transferred into E. coli Topo10. The recombinant plasmid was isolated from E. coli and digested with restriction enzymes NdeI and SpeI. The NdeI-SpeI DNA fragment was purified and ligated into NdeI/SpeI digested pET22b(+) expression vector (Novagen, Darmstadt, Germany). The resulting plasmid was transferred into E. coli, BL21 (DE3)LysS. pGEX4T-2 was used as a vector (Amersham Pharmacia Biotech, Piscataway, N.J.); the following primer pairs were also used: sense: 5′-GGATCCATGTTTCGTCGTCCAC (SEQ ID NO:8), and anti-sense: 5′-GAATTCTTAACCCCCCTTTACGG (SEQ ID NO:9). The restriction-enzyme sites, BamHI and EcOR1 were underlined. The recombinant plasmids were also transferred into E. coli.

### Example 4

### Purification of Omp and Antiserum Production

The BL21 (DE3)LysS strain containing the omp gene was grown in LB broth containing ampicillin (50 μg/mL) and chloramphenical (34 μg/mL). After OD₆₀₀ reached approximately 0.5, IPTG was added to a final concentration of 1 mM and the culture was grown for 4 h at 37° C. The cells were harvested and French pressed as previously described (Chang et al., “Recombinant OspA Protects Dogs Against Infection and Disease Caused by Borrelia burgdorferi,” Infect. Immun. 63:3543-3549 (1995), which is hereby incorporated by reference in its entirety). The protein samples were separated by SDS-polyacrylamide gel electrophoresis (SDS-PAGE). Proteins were visualized by staining with Coomassie blue and the Omp band was excised and mixed with an equal volume of PBS and was ground to homogeneity for rabbit injection. Polyclonal antiserum to Omp was raised in New Zealand White rabbits as previously described (Chang et al., “Recombinant OspA Protects Dogs Against Infection and Disease Caused by Borrelia burgdorferi,” Infect. Immun. 63:3543-3549 (1995), which is hereby incorporated by reference in its entirety).

### Example 5

### Fractionation of Cells

The fractionation of cells into portions enriched for inner and outer membranes was performed by sarkosyl differential solubilization as previously described (Drouet et al., “Characterization of an Immunoreactive Species-Specific 19-Kilodalton Outer Membrane Protein from Helicobacter pylori by Using a Monoclonal Antibody,” J. Clin. Microbiol. 29:1620-1624 (1991), which is hereby incorporated by reference in its entirety). Briefly, the H. bizzozeronii cell pellets were suspended in 0.01 MTris (pH 7.4) and ruptured by French press at 8,000 psi. The mixture was centrifuged at 5,000×g for 20 min to remove whole cells, and the supernatant was ultracentrifuged at 100,000×g for 1 h (Beckman Instruments Inc., Fullerton, Calif.). The pellet was suspended in sterile distilled water and added to 15 mL of 1% sodium lauryl sarcosinate (Sarkosyl; SIGMA, MO) in 7 mM EDTA and incubated at 37° C. for 20 min. The suspension was centrifuged at 100,000×g for 1 h and the pellet was suspended in water.

### Example 6

### SDS-PAGE and Western Blotting

The procedures for the SDS-PAGE and Western-blot analyses were as previously described (Chang et al., “Cloning, Sequencing and Expression of a Pasteurella haemolytica A1 Gene Encoding a PurK-Like Protein,” DNA Seq. 3:357-367 (1993); Chang et al., “Molecular Analysis of the Actinobacillus pleuropneumoniae RTX Toxin-III Gene Cluster,” DNA Cell Biol. 12:351-362 (1993), which are hereby incorporated by reference in their entirety). For identification of the expression of Omp, the rabbit anti-Lip18 anti-serum served as the primary antibody (1:500). The goat alkaline phosphatase-conjugated anti-rabbit IgG (KPL, Gaithersburg, Md.) was used as a secondary antibody (1:5,000). To determine whether the naturally infected dog sera contain anti-Lip18 antibodies, recombinant Lip18 proteins were used as antigen and subjected to SDS-PAGE and Western-blot analysis. Test sera (1:500) from naturally infected animals were used as a first antibody, followed by goat anti-dog or anti-cat IgG conjugated to alkaline phosphate (1:5,000) (KPL, Gaithersburg, Md.) as a second antibody.

### Example 7

### Nucleotide Sequence Accession Number

The DNA sequence of the outer membrane protein gene, also generally referred to herein as lip18, has been submitted to Genbank and assigned accession number AF330622.

### Example 8

### Cloning and Sequence of H. bizzozeronii lip18 Gene

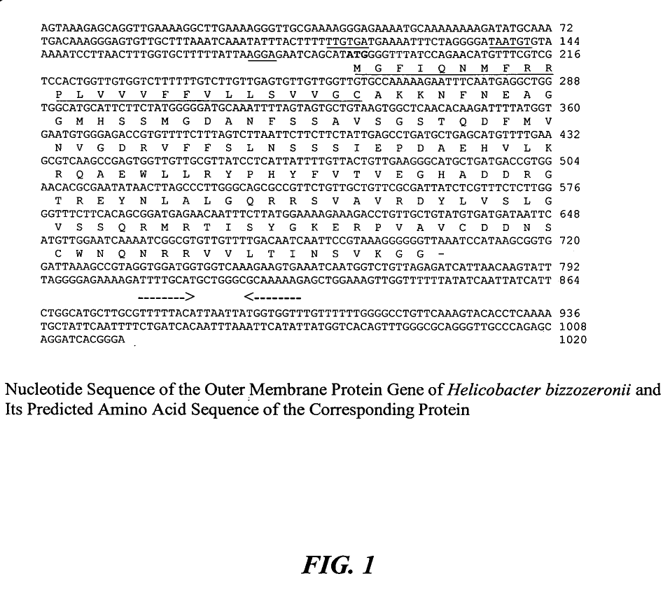

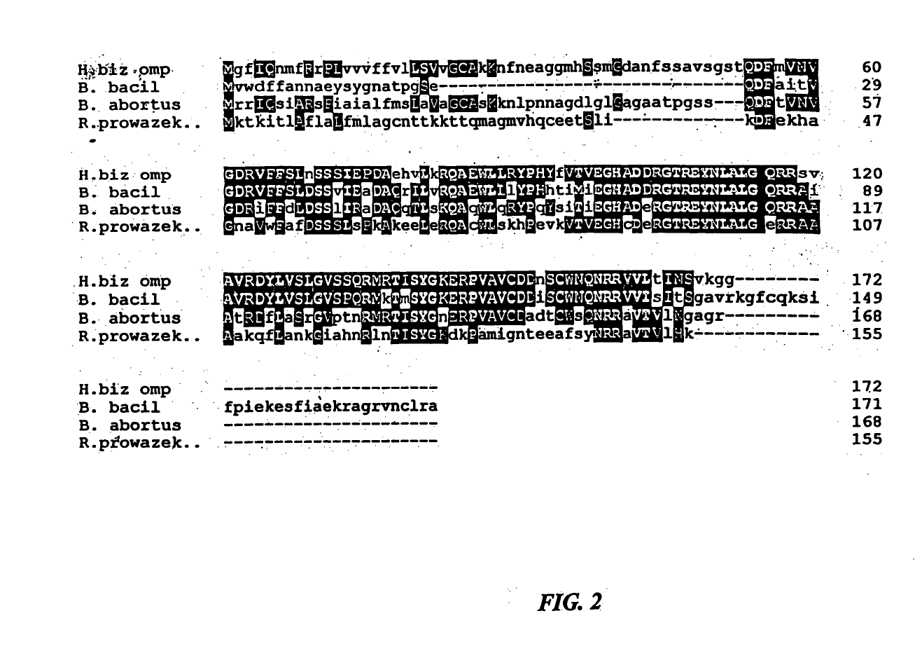

The H. bizzozeronii genomic library constructed in the phage vector λ-Zap II was screened. Five positive plaques were identified from 30,000 plaques and one of them was sequenced. It contains 1,020 bp, and one open reading frame (ORF) of 500 nucleotides was identified (FIG. 1). The ORF presumably encodes a Lip18 protein with predicted molecular mass 19,232 kD. The PI value of this Omp was predicted to be 9.15. A potential Shine-Dalgamo ribosome-binding site (AGGA) is from −10 to −16 of the start codon. There was a sequence, TAATGT, which is similar to the TATAAT consensus promoter sequence (−10 region) and one sequence, TTGTGA, (−35 region) proximal. A region of dyad symmetry, a possible rho-dependent transcription terminator (bases 806 to 827) was identified 100 bp downstream from the termination codon (FIG. 1). The amino acid sequence (aa) of Lip18 shows 57.1% homology to that of Brucella abortus (AAA59360), 56.7% to Bartonella bacilliformis 18.5 kD (AAA78948), and 55.2% to Agrobacterium tumefaciens (AAL44523). However, the sequence had less (22.1%) homology to Omp in H. pylori strains (NP223771 and U75869) (FIG. 2). FIG. 2 indicates that the N-terminal amino acid sequences were less homologous than the C-terminal among these species. For example, only eleven aa were identical between H. bizzozeronnii and B. bacilliformis within the first 46 aa peptides and four aa show identity between H. bizzozeronnii and H. pylori within the first 83 aa. However, Omp sequences showed higher homology at the C-terminal end of the polypeptides (FIG. 2).

### Example 9

### Expression of H. bizzozeronii Omp in Recombinant Clones and Western Blot Analysis

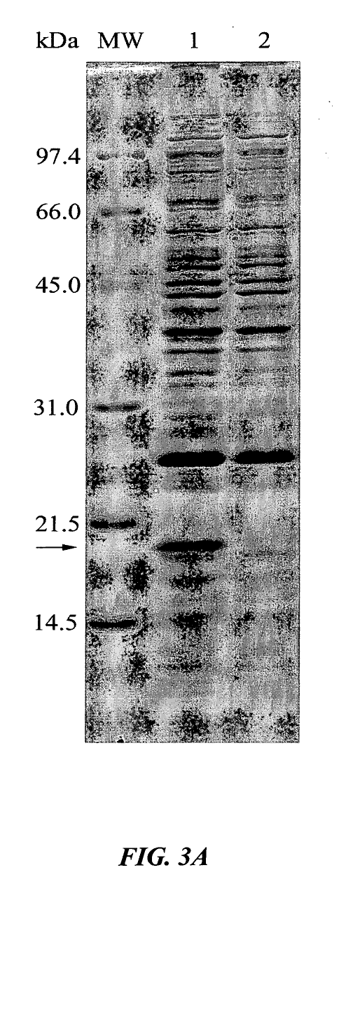

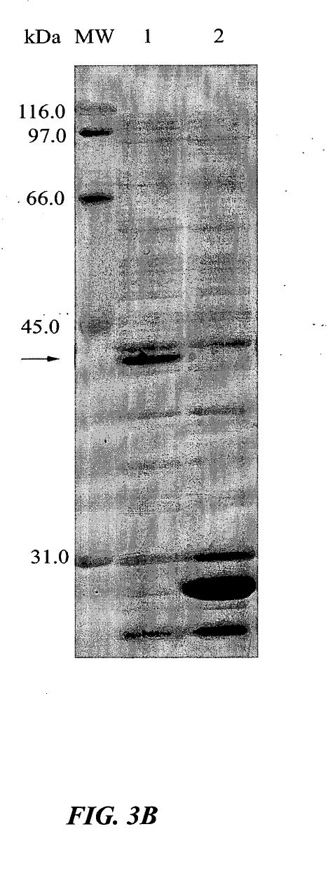

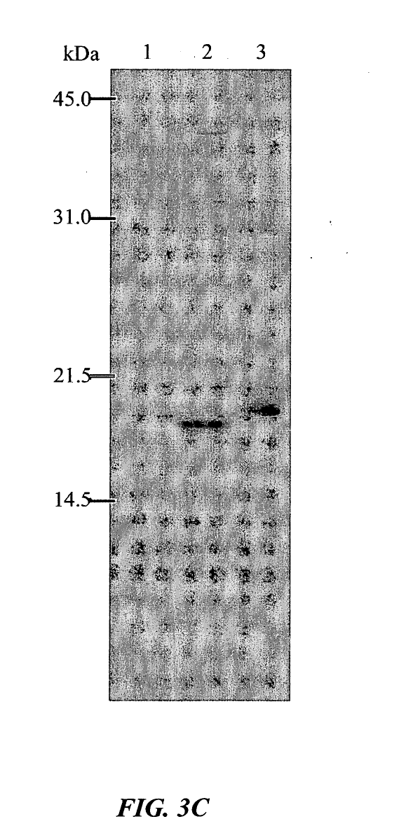

To express the Omp in E. coli, the omp gene was ligated into pET22 (b) and pGEX4T-2 and transferred into E. coli separately. SDS-PAGE and Western-blot analyses of whole-cell lysates of H. bizzozeronii and E. coli clones harboring the omp gene indicated that it was expressed with a molecular mass of approximately 18 kD (FIG. 3A, and 3C) or 43 kD glutathione S-transferase (GST)-fusion protein (FIG. 3B).

### Example 10

### Membrane Fractionation of the Lip18

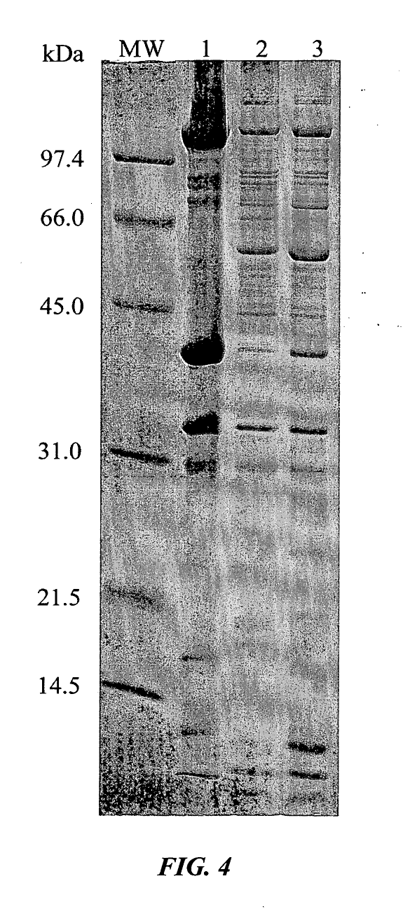

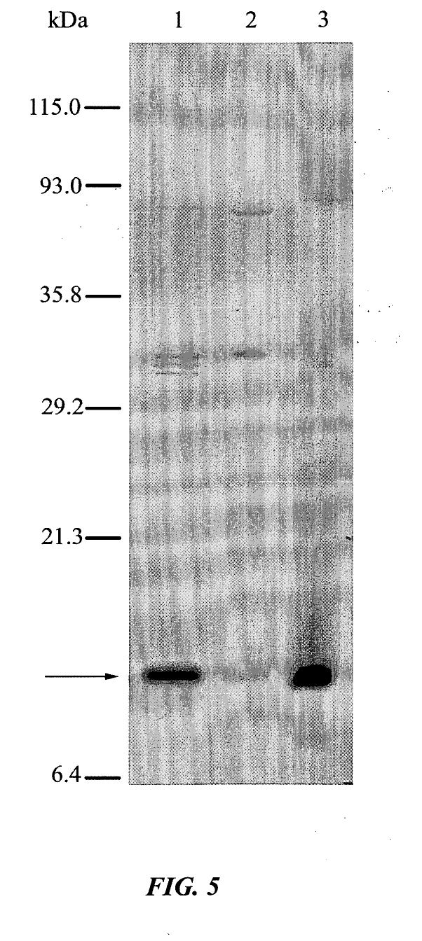

The outer-membrane fractions of H. bizzozeronii were isolated. Approximately nine outer-membrane polypeptide bands, with apparent molecular masses of 12, 18, 19, 34, 41, 85, 90, 92, and 100 kD were visualized by Coomassie-blue staining on a 12% SDS-PAGE (FIG. 4). Lip18 was mainly present in the sarkasyl insoluble fraction (outer membrane) and to a lesser extent in the soluble fraction (inner membrane). Western-blot analysis results indicated that Lip18 exists in the outer-membrane fraction (FIG. 5).

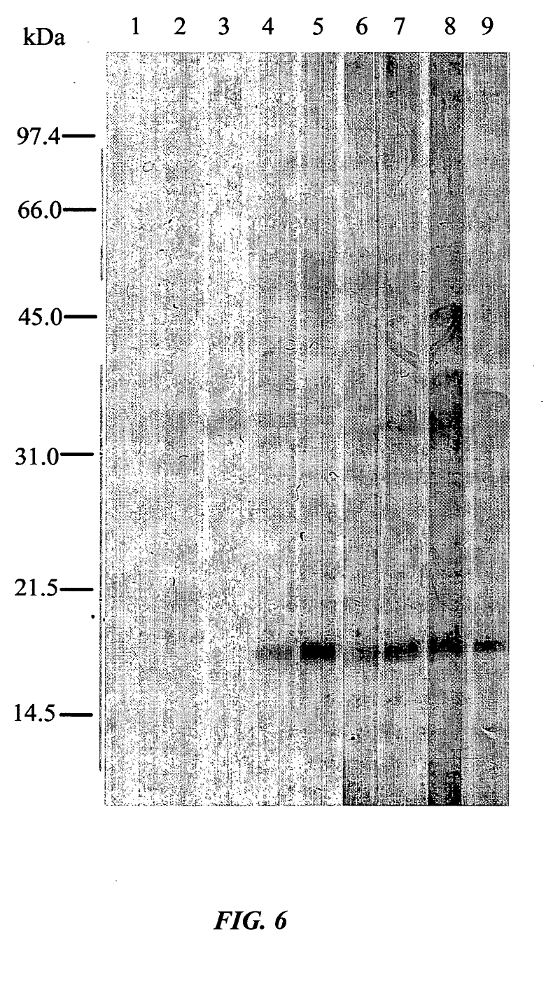

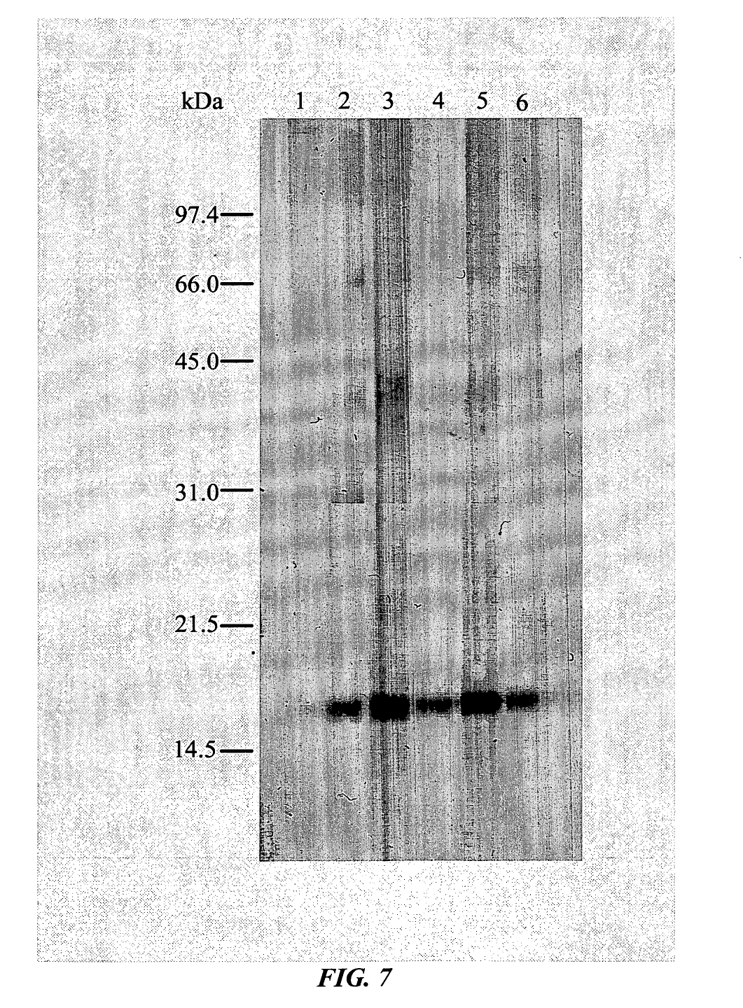

To evaluate whether sera from a naturally infected dog contains anti-Lip18 antibodies, naturally infected and uninfected dog and cat sera were subjected to Western-blot analysis. The results showed that partially purified rLip18 was recognized by the naturally infected (with H. bizzozeronii) dog and cat antisera, but not by the normal or the SPF dog sera (FIGS. 6 and 7). These data suggest in vivo expression of Lip18 and its involvement in canine and feline helicobacteriosis.

### Example 11

### Characterization of Lip18

An 18-kD outer-membrane lipoprotein (Lip18) was identified in H. bizzozeronii. Lip18 shows significant similarity to a peptidoglycan-associated lipoprotein found in several other pathogenic bacteria. This protein is of interest because one of the homologs (lipoprotein 20 of H. pylori) has been shown to induce a protective immune response (Keenan et al., “hmnune Response to an 18-Kilodalton Outer Membrane Antigen Identifies Lipoprotein 20 as a Helicobacter pylori Vaccine Candidate,” Infect. Immun. 68:3337-3343 (2000), which is hereby incorporated by reference in its entirety). In Haemophilus influenzae, another protein, P6, contains a surface-exposed epitope that can be recognized by monoclonal antibody (MAB)3B9, and P6 was able to induce protective immunity (Murphy et al., “The P6 Outer Membrane Protein of Nontypeable Haemophilus influenzae as a Vaccine Antigen,” J. Infect. Dis. 165:S203-205 (1992); Green et al., “Evaluation of Mixtures of Purified Haemophilus influenzae Outer Membrane Proteins in Protection Against Challenge with Nontypeable H. influenzae in the Chinchilla Otitis Media Model,” Infect. Immun. 61:1950-1957 (1993), which are hereby incorporated by reference in their entirety). P6 has 3B9 bind to a conformational determinant composed of two discontinuous regions, 87GNTDERGT94 and 147RR148 (Bogdan et al., “Mapping of a Surface-Exposed, Conformational Epitope of the P6 Protein of Haemophilus influenzae,” Infect. Immun. 63:4395-4401 (1995); Spinola et al., “The Conserved 18,000-Molecular-Weight Outer Membrane Protein of Haemophilus ducreyi has Homology to PAL,” Infect. Immun. 64:1950-1955 (1996), which are hereby incorporated by reference in their entirety). If histidine (His) and alanine (Ala) residues replaced the asparagine (Asn) and threonine (Thr) resisdues at 88 and 89, this P6 mutant still binds MAB3B9. In Lip18, there are two similar sequences, 100GHADDRGT107 and 160RR161. The spacing (53 aa) between the two regions is the same as in Haemophilus influenzae, H. bizzozeronii, and H. pylori (Kim et al., “Cloning and Characterization of a 22 kDa Outer-Membrane Protein (Omp22) from Helicobacter pylori,” Mol. Cells 10:633-641(2000), which is hereby incorporated by reference in its entirety).

The predicted N-terminal amino acid sequence of H. bizzozeronii had features typical of a lipoprotein signal peptide and a signal peptidase-II processing site (Bogdan et al., “Mapping of a Surface-Exposed, Conformational Epitope of the P6 Protein of Haemophilus influenzae,” Infect. Immun. 63:4395-4401 (1995); Spinola et al., “The Conserved 18,000-Molecular-Weight Outer Membrane Protein of Haemophilus ducreyi has Homology to PAL,” Infect. Immun. 64:1950-1955 (1996), which are hereby incorporated by reference in their entirety). A mature lipoprotein is yielded after lipid modification and proteolytic processing, and is localized into either the inner or the outer membrane (Wu et al., “Biogenesis of Lipoprotein in Bacteria,” Curr. Top. Microbiol. Immunol. 125:127-157 (1986), which is hereby incorporated by reference in its entirety). Bacterial lipoprotein has a consensus sequence of Leu-X-Y-Cys (Wu et al., “Biogenesis of Lipoprotein in Bacteria,” Curr. Top. Microbiol. Immunol. 125:127-157 (1986), which is hereby incorporated by reference in its entirety). X and Y are neutral small amino acids. However, many sequence results indicate that the Leu residue can be replaced by several other amino acids. In H. bizzozeronii Lip18, leucine was replaced by valine. The same was shown in B. abortus 16.5-kD lipoprotein. Substitution occurred also in H. pylori and E. coli lipoproteins: isoleucine replaced leucine (Chen et al., “Nucleotide Sequence of the Gene for the Peptidoglycan-Associated Lipoprotien of Escherichia coli K12,” Eur. J. Biochem. 163:73-77 (1987); Kostrzynska et al., “Molecular Characterization of a Conserved 20-Kilodalton Membrane-Associated Lipoprotein Antigen of Helicobacter pylori,” J. Bacteriol. 176:5938-5948 (1994), which are hereby incorporated by reference in their entirety). Similar to the 18.5-kD Omp in Bartonella bacilliformis, the Omp of the present invention could be a peptidoglycan-associated lipoprotein with a 24-aa signal sequence. Even though Gly and Phe at the second and third positions of the N-termini were not positively charged, the Gin at the fifth position was a positively charged residue. The signal peptide composes Val-Val-Gly-Cys that is identical to the lipoprotein signal peptide sequence of H. pylori J99 strain (NP223771). This sequence is required for processing by signal peptidase II (Wu et al., “Biogenesis of Lipoprotein in Bacteria,” Curr. Top. Microbiol. Immunol. 125:127-157 (1986), which is hereby incorporated by reference in its entirety). Leucine is replaced by alanine, isoleucine, or valine in several bacteria species (Chen et al., “Nucleotide Sequence of the Gene for the Peptidoglycan-Associated Lipoprotien of Escherichia coli K12,” Eur. J. Biochem. 163:73-77 (1987); Kim et al., “Cloning and Characterization of a 22 kDa Outer-Membrane Protein (Omp22) from Helicobacter pylori,” Mol. Cells 10:633-641 (2000), which are hereby incorporated by reference in their entirety).

For the localization of the lipoprotein, it has been suggested that the amino acid at the +2 position of the N-terminal of the mature polypeptide is a sorting signal (Yamaguchi et al., “A Single Amino Acid Determinant of the Membrane Localization of Lipoprotiens in E. coli,” Cell 53:423-432 (1988), which is hereby incorporated by reference in its entirety). Typically, serine at this position indicated an outer-membrane protein, whereas arginine indicated an inner-membrane protein. This sorting method is based on E. coli; however, there are exceptions to this rule (Hayashi et al., “Lipoproteins in Bacteria,” J. Bioenerg. Biomembr. 22:451-471 (1990), which is hereby incorporated by reference in its entirety). In H. pylori KCTC0217BP, although Lys was at the +2 position, the protein was located on the outer membrane (Kim et al., “Cloning and Characterization of a 22 kDa Outer-Membrane Protein (Omp22) from Helicobacter pylori,” Mol. Cells 10:633-641 (2000), which is hereby incorporated by reference in its entirety). Lip18 at N-2 had neither aspartic acid nor serine, therefore, it is possibly not an inner-membrane protein but an outer-membrane protein. Results also showed that the native Lip18 was detected mainly in the outer membrane and to a lesser extent in the inner membrane of H. bizzozeronii. Part of the reason is that in Helicobacter spp., the inner and outer membrane proteins are are not separated totally by currently available methods (Kostrzynska et al., “Molecular Characterization of a Conserved 20-Kilodalton Membrane-Associated Lipoprotein Antigen of Helicobacter pylori,” J. Bacteriol. 176:5938-5948 (1994); Kim et al., “Cloning and Characterization of a 22 kDa Outer-Membrane Protein (Omp22) from Helicobacter pylori,” Mol. Cells 10:633-641 (2000), which are hereby incorporated by reference in their entirety).

Whereas sera obtained from dogs or cats naturally infected with Helicobacter spp. contained antibodies that bind to recombinant H. bizzozeronii Lip18, those from SPF dogs and cats did not. This indicates that Lip18 was expressed during infection.

In conclusion, a Lip18 in H. bizzozeronii was identified that is highly similar to other bacterial lipoproteins, especially at the C-terminus, and is a possible vaccine candidate. Furthermore, the Lip18 had low similarity for the first 240 nucleotides from the N-terminus as compared to that of H. pylori. This part of the DNA sequence may be useful for PCR differential diagnosis of H. bizzozeronii and H. pylori.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. An isolated nucleic acid molecule encoding an outer membrane protein of Helicobacter bizzozeronii.

2. An isolated nucleic acid molecule according to claim 1, wherein said nucleic acid molecule encodes a protein or polypeptide having an amino acid sequence of SEQ ID NO: 2; has a nucleotide sequence of SEQ ID NO: 1; or has a nucleotide sequence comprising at least 20 contiguous nucleic acid residues that hybridize to a nucleic acid having a nucleotide sequence corresponding to SEQ ID NO: 1 under stringent conditions comprising 50 percent formamide at 42 degrees Celsius.

3. An isolated protein or polypeptide encoded by a nucleic acid molecule according to claim 1.

4. An isolated protein or polypeptide according to claim 3, wherein said protein or polypeptide is recombinant.

5. An isolated protein or polypeptide according to claim 3, wherein said protein or polypeptide is purified.

6. A method of vaccinating mammals against onset of disease resulting from infection by Helicobacter bizzozeronii comprising:
administering to mammals an effective amount of at least one isolated protein or polypeptide according to claim 3.

7. The method according to claim 6, wherein said administering is oral, intradermal, intramuscular, intraperitoneal, intravenous, subcutaneous, or intranasal.

8. A method of vaccinating mammals against onset of disease caused by infection of Helicobacter bizzozeronii comprising:
administering to mammals an effective amount of at least one isolated nucleic acid molecule according to claim 1.

9. The method according to claim 8, wherein said administering is oral, intradermal, intramuscular, intraperitoneal, intravenous, subcutaneous, or intranasal.

10. A recombinant DNA expression system comprising an expression vector into which is inserted a heterologous nucleic acid molecule according to claim 1.

11. A recombinant DNA expression system comprising an expression vector into which is inserted a heterologous nucleic acid molecule according to claim 2.

12. A recombinant DNA expression system according to claim 10, wherein said heterologous nucleic acid molecule is inserted into said vector in proper orientation and correct reading frame.

13. A host cell incorporating a heterologous nucleic acid molecule according to claim 1.

14. A host cell according to claim 13, wherein said nucleic acid molecule is inserted into a recombinant DNA expression system comprising an expression vector.

15. A vaccine for preventing onset of disease in mammals infected by Helicobacter bizzozeronii comprising:
an isolated protein or polypeptide according to claim 3; and
a pharmaceutically-acceptable carrier.

16. A method of vaccinating mammals against onset of disease resulting from infection by Helicobacter bizzozeronii comprising:
administering to mammals an effective amount of the vaccine according to claim 15.

17. The method according to claim 16, wherein said administering is oral, intradermal, intramuscular, intraperitoneal, intravenous, subcutaneous, or intranasal.

18. An isolated antibody or binding portion thereof raised against a protein or polypeptide according to claim 3.

19. An isolated antibody or binding portion according to claim 18, wherein said antibody is monoclonal or polyclonal.

20. A method of passively immunizing mammals infected with Helicobacter bizzozeronii comprising:
administering an effective amount of said antibody or binding portion thereof according to claim 18 to mammals infected with Helicobacter bizzozeronii.

21. A method according to claim 20, wherein said administering is oral, intradermal, intramuscular, intraperitoneal, intravenous, subcutaneous, or intranasal.

22. A composition for passively immunizing mammals infected with Helicobacter bizzozeronii comprising:
an isolated antibody or binding portion thereof according to claim 18; and
a pharmaceutically-acceptable carrier.

23. A composition according to claim 22, wherein said antibody is monoclonal or polyclonal.

24. A method of passively immunizing mammals infected with Helicobacter bizzozeronii comprising:
administering an effective amount of said composition according to claim 22 to mammals infected with Helicobacter bizzozeronii.

25. A method according to claim 24, wherein said administering is oral, intradermal, intramuscular, intraperitoneal, intravenous, subcutaneous, or intranasal.

26. A method for detection of Helicobacter bizzozeronii in a sample of tissue or body fluids comprising:
providing a protein or polypeptide according to claim 3 as an antigen;
contacting the sample with the antigen; and
detecting any reaction which indicates that Helicobacter bizzozeronii is present in the sample using an assay system.

27. The method according to claim 26, wherein the assay system is selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

28. A method for detection of Helicobacter bizzozeronii in a sample of tissue or body fluids comprising:
providing an antibody or binding portion thereof according to claim 18;
contacting the sample with the antibody or binding portion thereof; and
detecting any reaction which indicates that Helicobacter bizzozeronii is present in the sample using an assay system.

29. A method according to claim 28, wherein said antibody is monoclonal or polyclonal.

30. A method for detection of Helicobacter bizzozeronii in a sample of tissue or body fluids comprising:
providing a nucleotide sequence of the nucleic acid molecule according to claim 1 as a probe in a nucleic acid hybridization assay;
contacting the sample with the probe; and
detecting any reaction which indicates that Helicobacter bizzozeronii is present in the sample.

31. A method for detection of Helicobacter bizzozeronii in a sample of tissue or body fluids comprising:
providing a nucleotide sequence of the nucleic acid molecule according to claim 1 as a probe in a gene amplification detection procedure;
contacting the sample with the probe; and
detecting any reaction which indicates that Helicobacter bizzozeronii is present in the sample.
